(19) United States
(12) Patent Application Publication
(10) Pub. No.: US 2013/0252324 A1
(43) Pub. Date: Sep. 26, 2013

(54) SYSTEM FOR ESTABLISHING SYNERGISM BETWEEN RENEWABLE ENERGY PRODUCTION AND FOSSIL ENERGY PRODUCTION

(71) Applicant: Charles Bliss, Springfield, VA (US)

(72) Inventor: Charles Bliss, Springfield, VA (US)

(21) Appl. No.: 13/901,008

(22) Filed: May 23, 2013

Related U.S. Application Data
(62) Division of application No. 13/407,976, filed on Feb. 29, 2012.

(51) Int. Cl. C12M 1/00

(52) U.S. Cl. CPC C12M 21/02
USPC 435/292.1

(57) ABSTRACT

A method and system for synergism between a renewable energy system and a fossil energy system for the production of marketable energy forms such as electricity, methanol, petroleum-equivalent transportation fuels, urea fertilizer, and industrial hydrogen. Depending on configuration, the invention relates either to fossil-energy-assisted, renewable energy production or to renewable-energy-assisted, fossil energy production. The fossil energy source is coal, hydrocarbon fuel oil, or natural gas. The renewable energy is from the cultivation of microalgae and the extraction of its oil content, production of methane, and coincidentally-produced oxygen. The products and by-products from fossil energy production are used for the cultivation of algae and the products and by-products from algae cultivation are used to reduce the consumption of fossil energy in energy production. The synergism is the interchange of these products and by-products such that the net marketable energy production results in maximizing the use of renewable (solar) energy, minimizing the use of fossil fuel, and minimizing emissions of carbon dioxide to the atmosphere.

### TECHNICAL FIELD OF THE INVENTION

The present invention relates generally to the synergism between renewable energy and fossil energy and more particularly to renewable energy embodied as the cultivation of algae and to fossil energy embodied as the use of coal and liquid hydrocarbon fuel oils.

### BACKGROUND OF THE INVENTION

One of the results of the industrial revolution of the past two centuries has been a 180-degree change from practically total global reliance on renewable energy to practically total global reliance on fossil energy, with the consequence of elevated carbon dioxide content in the atmosphere and its potential impact on deleterious climate change. Worldwide concern with this phenomenon has resulted in major international and national research and development programs to capture carbon dioxide from dilute streams in fossil energy production systems to be followed by permanent sequestration of the captured gas in secure underground geological formations, to the extent that this captured gas does not have industrial uses such as enhancing the recovery of petroleum oil from mature underground formations.

One promising candidate for generating a market for captured carbon dioxide lies in the cultivation of selected algae strains, especially microalgae that have high content of combustible oil. Considerable intensive worldwide research and development activity currently exists aimed at two modes of algae cultivation: one in open ponds and the other in enclosed structures known as photobioreactors. The open-pond mode can be characterized as requiring large land areas in terms of their output, subject to adverse environment impact in the form of invasion by unwanted algae species and the effects of wind, evaporation, and storms. Most importantly the ponds lose significant quantities of unreacted carbon dioxide to the atmosphere and the oxygen produced from the photosynthesis process is not recoverable for use. These disadvantages are avoided by the use of enclosed photobioreactors to grow algae.

Current research and development work in algae cultivation is largely based on the use of a diluted form of carbon dioxide by directly transporting combustion gases from fossil energy power generating systems to the algae systems. Plant growth rates accelerate significantly when the level of carbon dioxide in the growth atmosphere increases. Emerging development in fossil-energy based power generating systems, notably in coal gasification and in oxyfuel combustion promise by-product streams of virtually 100% content (neat) carbon dioxide as contrasted with the about 13-15% carbon dioxide content streams from conventional combustion emission gases. The availability of neat carbon dioxide promises major reductions in land area required for algae cultivation when photobioreactors are employed, while greatly reducing the loss to the atmosphere of supplied carbon dioxide and allowing the recovery of the oxygen produced from the photosynthesis process.

### SUMMARY OF THE INVENTION

The present invention is based on fossil energy power generating systems that produce neat, or near neat, carbon dioxide and use closed-system photobioreactors that combine solar energy input (insolation) with artificial illumination.

The present invention involves synergism between a renewable energy form and a fossil energy form for the production of marketable energy forms such as electricity, methanol, petroleum-equivalent transportation fuels, urea fertilizer, and industrial hydrogen. Depending on configuration, the invention may be considered either as fossil-energy-assisted, renewable energy production or as renewable-energy-assisted, fossil energy production. The fossil energy component may be coal, petroleum fuel oil, or natural gas based. The renewable energy component is the cultivation of microalgae and the extraction of its oil content, coincidentally-produced oxygen, and use of spent algae to produce methane. The invention uses products and by-products from fossil energy production for the cultivation of algae and uses products and by-products from algae cultivation to reduce the consumption of fossil energy in energy production. The synergism is the interchange of these products and by-products such that the net marketable energy production results in maximizing the use of renewable (solar) energy, minimizing the use of fossil fuel, and minimizing emissions of carbon dioxide to the atmosphere.

The present invention relates generally to the production of marketable energy forms from either, or both, renewable energy and fossil energy sources, and more particularly to renewable energy embodied as the cultivation and processing of algae and to fossil energy embodied as the use of coal and liquid and gaseous fossil fuels. Renewable energy is embodied in the present invention as the cultivation and processing of algae for use of its oil content and for use of its by-product production of almost pure oxygen. Also embodied in the present invention is the alternative production of almost pure methane, which is of pipeline quality. Fossil energy is embodied as the primary use of by-product carbon dioxide for algae cultivation, the use of all ranks of coal, but also of the use of liquid and gaseous fossil fuels such as petroleum fuel oils and natural gas, and its potential to supply temperature-controlled makeup water to the algae cultivation and processing system. Natural gas is embodied in its use for power generation in both a direct combustion mode and in a combined cycle mode.

This invention then addresses linking inputs and the products from renewable energy with those of fossil energy such as to result in a combined system that maximizes the use of solar energy, minimizes the use of fossil energy, and minimizes the emissions of carbon dioxide to the atmosphere.

Accordingly, the present invention is directed to a system to process carbon dioxide for algae cultivation that includes a photobioreactor array for using the carbon dioxide from a fossil energy production system to produce algae both from solar energy and artificial illumination, separately or combined, that recovers the oil content of the algae, that provides almost pure oxygen as a by-product stream, and that distributes the by-product carbon dioxide stream to the photobioreactor array.

The present invention is also directed to the production of a by-product carbon dioxide stream through the capture of carbon dioxide produced from the use of a fossil fuel in any of three modes for energy production that involve either direct combustion or gasification in which excess carbon is produced as by-product carbon dioxide. The gasification of a solid or liquid fuel produces a mixture of carbon monoxide and hydrogen (synthesis gas) that can be further processed to a variety of energy products, including electricity, with excess carbon being produced as by-product carbon dioxide. The combustion of a solid or liquid fossil fuel in an “oxyfuel combustion” mode produces practically all of the carbon as by-product carbon dioxide because of the circulation of the combustion gases to replace the nitrogen in the air for the combustion of the fossil fuel. In conventional combustion in which a specific solvent extracts carbon dioxide as a pure stream, practically of the carbon dioxide can be extracted as a by-product carbon dioxide because of the inclusion of a solvent extraction process. In all three modes, by-product oxygen from algae cultivation can be used to enhance the thermal efficiency of the gasification or combustion process.

### BRIEF DESCRIPTION OF THE DRAWINGS

The structure, operation, and advantages of the present invention will become further apparent upon consideration of the following description taken in conjunction with the accompanying figures (FIGs.). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

FIG. 1 is a schematic view of a system for the cultivation of algae and for processing the algae for the production of its oil, of by-product oxygen, and of the processing of oil-free algae for the production of methane, in accordance with the present invention.

FIG. 1A is a chart that illustrates relationships between residence time and the percent of completed reactions for algae cultivation, in accordance with the present invention.

FIG. 2 shows a perspective configuration of a tube in a photobioreactor array in which the photosynthesis reactions occur, in accordance with the present invention.

FIG. 2A shows a top view of the tube in a photobioreactor, in accordance with the present invention.

FIGS. 3A, 3B and 3C are front end views of three embodiments of the tube concentrically placed within a tube of a photobioreactor complete with fluorescent or “neon” light emitting elements, in accordance with the present invention.

FIG. 4 is a front end view of an embodiment of the tube within a photobioreactor complete with light emitting diodes (LEDs), in accordance with the present invention.

FIG. 5 is a “footprint” configuration of the modules in a photobioreactor array such as is shown in FIG. 1, in accordance with the present invention.

FIG. 6 shows the linkage configuration between an algae cultivation and processing system and a typical coal-gasification based energy system, in accordance with the present invention. In another embodiment coal may be substituted by a liquid hydrocarbon fuel.

FIG. 7 shows the linkage configuration between an algae cultivation and processing system and a typical pulverized-coal based electricity production system employing Oxyfuel Combustion, in accordance with the present invention. In another embodiment coal may be substituted by a liquid or gaseous hydrocarbon fuel.

FIG. 8 shows the linkage configuration between an algae cultivation and processing system and a typical pulverized-coal based electricity production system employing enhanced oxygen combustion mode, in accordance with the present invention.

### DETAILED DESCRIPTION OF THE PREFERRED EMBODIMENTS

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

In the drawings accompanying the description that follows, often both reference numerals and legends (labels, text descriptions) will be used to identify elements. If legends are provided, they are intended merely as an aid to the reader, and should not in any way be interpreted as limiting.

The present invention is generally directed to the linkage between an Algae Cultivation and Processing system and three modes for Fossil Energy Production. Accordingly, the following description is clarified by two subheadings: Algae Cultivation and Processing and Fossil Energy Production. The Algae Cultivation and Processing System is common to each of the three Fossil Energy Production modes set forth herein below.

### Algae Cultivation and Processing System

A preferred embodiment involves the cultivation of oil-bearing algae to provide from its products fuels and oxygen to complement coal and liquid hydrocarbon fuels for either gasification to produce electrical power or a range of commodity energy products such as petroleum-equivalent distillate fuels, methanol, and industrial hydrogen, or, to complement coal and liquid and gaseous hydrocarbon fuels in Oxyfuel Combustion or in conventional combustion to produce electrical power. In a combined-cycle mode, the algae oil can be delivered and combusted in the heat-recovery, steam generator to increase the supply of superheated steam to the turbo-generator to produce electricity. The cultivation of algae employs captured carbon dioxide from the gasification or combustion processes.

A major issue that arises in a prospective commercial application of the preferred embodiment is the fact that captured carbon dioxide is produced during a 24 hour 7 days a week continuous operating schedule while an algae cultivation system can only cultivate algae during daylight hours. If a nighttime market exists for captured carbon dioxide, such as in enhanced oil recovery, this issue disappears. Otherwise, systems are necessary to store nighttime carbon dioxide production for use during the daytime hours in order to avoid its disposal into the atmosphere.

Conventional techniques are readily available to accommodate this dichotomy. Carbon dioxide captured during nighttime can be stored under pressure in underground caverns, which might be available nearby, for release during the daytime.

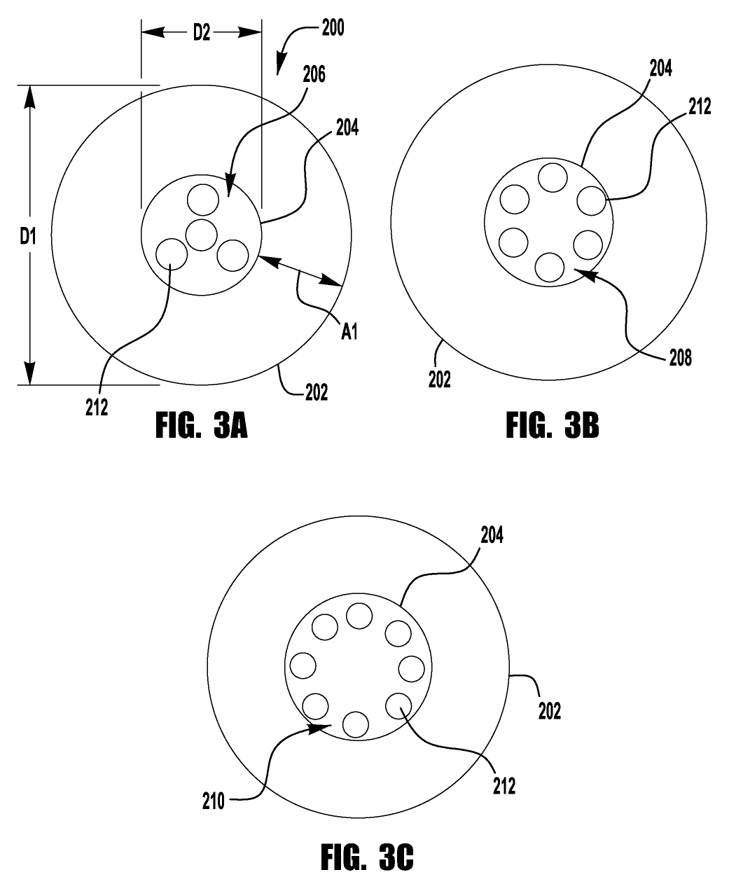

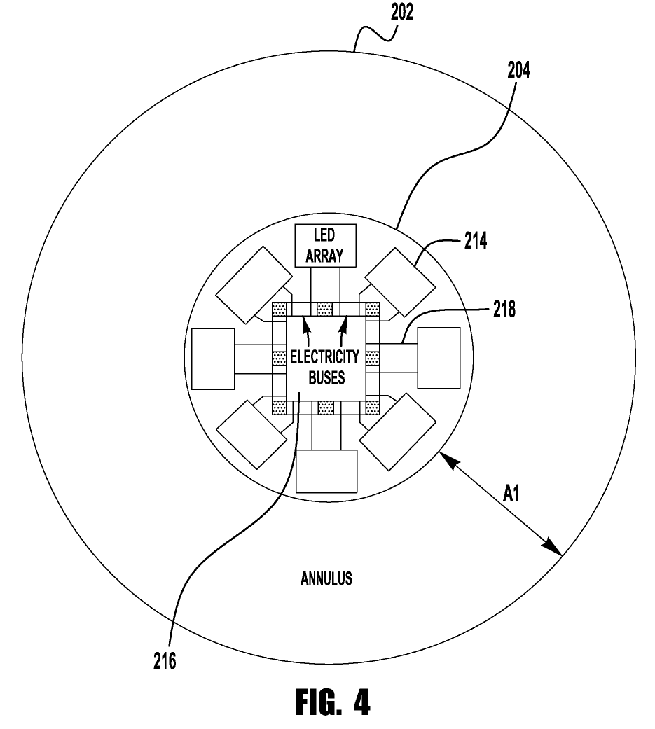

Alternatively, captured carbon dioxide could be used to manufacture dry ice during the night, which can then be evaporated during the daylight hours. It could also be liquefied and stored under pressure for evaporation during the daytime. But these methods of storing carbon dioxide generated during the night for use during daylight hours by the algae cultivation system not only require the algae cultivation installations to have about twice the production capacity than it would otherwise have under a 24 hour 7 days a week operating schedule, but also require that the installed system operates only about half of the time. Cost attractiveness would be significantly diminished not only because of the low load factor in cultivation but also in the cost of interim storage for captured carbon dioxide and its liberation. These conventional techniques can be avoided by the use of artificial illumination embodied in this invention as further described below in connection with FIGS. 3A, 3B, 3C, and 4.

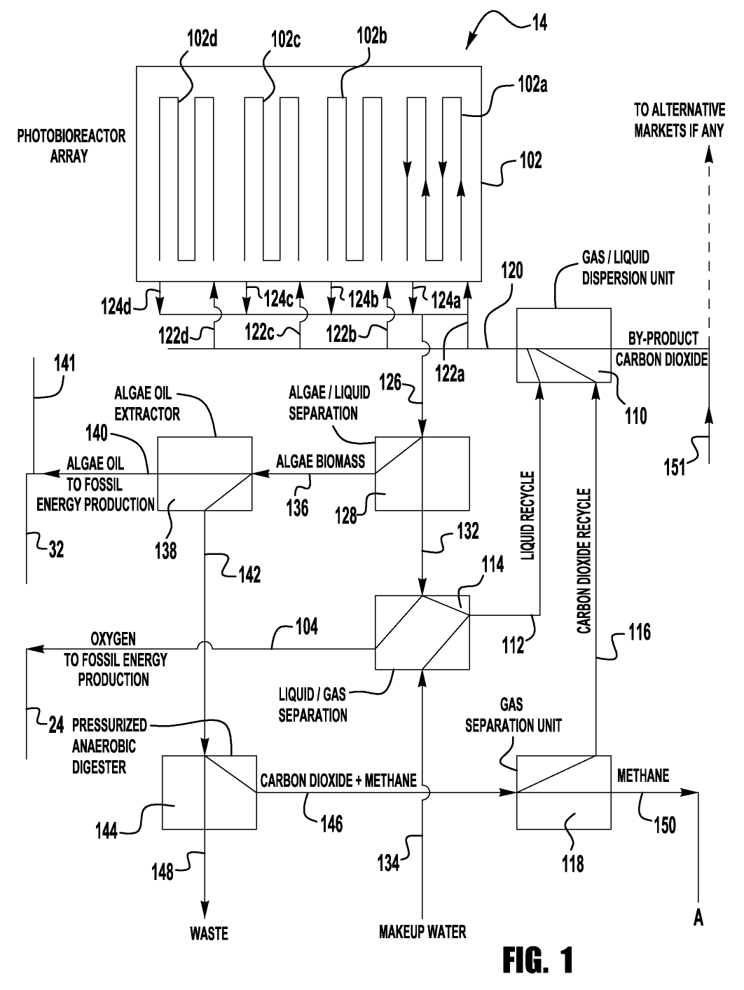

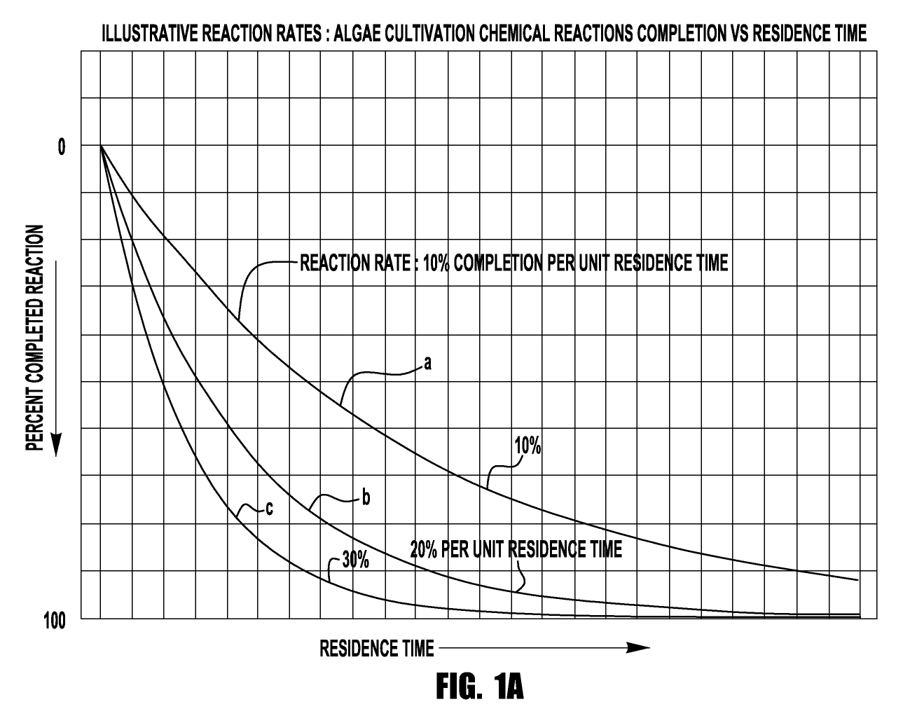

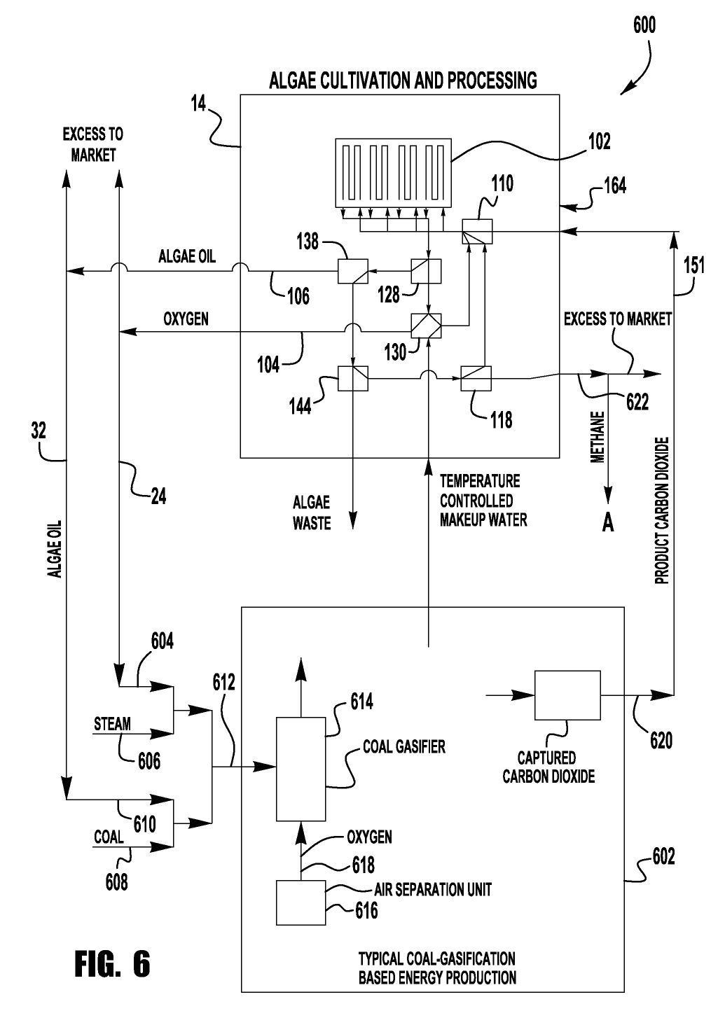

As shown in FIG. 1, the present invention is directed to a system 14 to continuously process carbon dioxide 151 in an algae cultivation and processing plant and simultaneously produce an algae-oil product 140 together with a by-product stream of practically pure oxygen 104. An embodiment is also directed as an option to produce an almost pure stream of methane 150. Systems 600, 700, and 800 as shown in FIGS. 6, 7, and 8 serve to support synergism between algae cultivation and processing and three modes of fossil energy production which is further described below in connection with FIGS. 6, 7, and 8.

The disclosed system 14 utilizes any proportion up to 100% of the carbon dioxide 151 supplied during a 24-hour period by any of the three modes of fossil energy generation depending on the availability of alternative markets for the carbon dioxide 151, such as in enhanced production of petroleum crude oil or in the manufacture of “dry ice”. This invention embodies the utilization of by-product, or captured, carbon dioxide produced during night time operation of any of the three modes of fossil energy production by incorporating facilities for providing artificial illumination to substitute for the absence of solar radiation (insolation) as described below.

Current research and development in the cultivation of selected species of algae has clearly shown the potential for the production of a fuel oil that in almost all aspects is equivalent, if not superior, to a petroleum-based residual fuel oil, such as No. 6 fuel oil. Algae fuel oil is sulfur and mineral-ash free, but since it is an oxygenated triglyceride, its heating value is slightly lower than No. 6 fuel oil. In addition, algae fuel oil can readily be converted to higher-valued biodiesel by a commercially-available esterification system. Algae cultivation requires carbon dioxide, water, and the photons of solar energy. Continuous algae production from available carbon dioxide can be accomplished in closed systems within photobioreactors. In a preferred embodiment, as schematically illustrated in FIG. 1, nighttime operation of the Algae Cultivation and Processing system 14 can be accomplished utilizing artificial illumination to substitute for solar illumination by providing light emitting elements within a photobioreactor (as described below).

The schematic diagram in the algae cultivation and processing system 14 of FIG. 1 illustrates how algae oil could be produced from a photobioreactor array 102. The balance of the algae cultivation and processing system 14, includes a recovery system 110, 114,118,128, and 138, which essentially recovers the oil content of the algae, provides recycle streams, distributes the captured carbon dioxide from line 151 to the photobioreactors 102, provides a by-product oxygen stream through line 104 and, optionally, digests the oil-free algae biomass 144 to produce carbon dioxide for recycle and methane 118.

The selection of the algae species for maximum oil production, the effect of artificial illumination in the absence of insolation at night, and its effect on complementing insolation during the day, are important factors. The Algae Cultivation and Processing system 14 shown in FIG. 1 addresses these factors and accepts the by-product carbon dioxide production through line 151 for the production of algae in the photobioreactor arrays 102, from which algae oil is produced by algae/liquid separation 128, algae-oil extraction 138 through line 140, and by-product oxygen by liquid/gas separation 114 through line 104. Optionally, it can also process oil-free algae biomass 142 by anaerobic digestion 144 to produce by-product methane 150 and carbon dioxide 116, which is recycled internally to the photobioreactor array 102.

The photobioreactor array 102 enables the cultivation of algae in a configuration of photobioreactor modules 102a, 102b, 102c, 102d, (102a-102d). Each module 102a-102d is designed to provide the residence time for optimized production of appropriately-selected algae species under conditions of essentially 100% carbon dioxide input and the presence of enzyme catalysts that maximize the oil content in the algae produced.

While the photobioreactor array 102 is shown with four modules 102a-102d, it's within the terms of the preferred embodiment to use any desired number of modules. The length of each module 102a-102d, the cross-sectional area for flow, and the volumetric flow rate for the reactants flowing through a module determine the residence time of the reactants during which the algae cultivation reactions proceed to the desired level of completion. Since each module 102a-102d can accommodate a predetermined quantity of carbon dioxide, the total number of modules required for Algae Cultivation and Processing system 14 equals the total amount of captured carbon dioxide available divided by the amount of carbon dioxide that each module can utilize. It is also within the terms of the invention to use a plurality of interconnected photobioreactor arrays 102 as shown in FIG. 1.

Further, as shown in FIG. 1, a gas/liquid dispersion unit 110 receives recycled liquid (after the algae has been harvested) containing the enzyme catalyst and the makeup water through lines 134 and 112 from a liquid/gas separation device 114. The gas/liquid dispersion unit 110 receives recycled recovered carbon dioxide (when anaerobic digestion 144 is incorporated through a line 116 from a gas separation unit 118, which separates the methane and carbon dioxide stream 146 produced from anaerobic digestion 144, together with by-product carbon dioxide 151. The by-product carbon dioxide 151 plus the recycled recovered carbon dioxide is received and disbursed as micro-bubbles within the liquid recycle and makeup water 110 either in bulk or at the entrance to each module 102a-102d. The gas/liquid dispersion unit 110 directs the by-product carbon dioxide plus the recycled recovered carbon dioxide as micro-bubbles within the liquid recycle and makeup water into a line 120 and then into lines 122a, 122b, 122c and 122d which are the inlet lines of modules 102a-102d, respectively, of the photobioreactor 102.

An effluent or solution of liquid and algae exit the modules 102a-102d through lines 124a, 124b, 124c, and 124d (124a-124d), respectively, and flow through a line 126 into an algae/liquid separation device 128 where the algae in the discharge from the photobioreactor modules 102a-102d is harvested by filtration (or alternatively by an electroporation process) to separate the algae from the recycled liquid.

In the device 114, the recycled water is received through line 132 from the algae/liquid separation device 128 and the dissolved and entrained gases (primarily oxygen) are flashed off for return through line 104 to the three modes of fossil energy production. Makeup water is added through line 134 and the recycled liquid mixture is sent through line 112 to the gas/liquid dispersion unit 110.

The algae biomass is directed from the algae/liquid separation device 128 through a line 136 into an algae oil extractor 138 where the algae biomass is subjected to removal of its algae oil content through a combination of pressing and solvent extraction. The latter step may be unnecessary if the algae oil has already been extracted through electroporation. The algae oil is then removed from the algae oil extractor 138 through line 140 and directed through a line 32 to be recycled to the fossil-fuel based energy production systems 600, 700 or 800 as shown in FIGS. 6, 7 and 8 or through line 141 to be sold to available markets.

The algae biomass residue can be removed from the algae oil extractor 138 through line 142 and delivered to a pressurized anaerobic digester 144 in which it produces a mixture of carbon dioxide and methane. This mixture is directed through a line 146 to the gas separation unit 118. The waste is directed through line 148 for disposal. The anaerobic digestion is conducted at elevated pressure to recover the carbon and hydrogen content of the oil-free algae biomass as carbon dioxide and methane. From here, the carbon dioxide can be recycled to the gas/liquid dispersion unit 110 through line 116. The residue, reduced in volume, is discarded through line 148.

FIG. 1A represents an illustrative relationship between residence times (along the horizontal axis) for the chemical reactions to complete and percentage of completed reactions (along the vertical axis). Three arbitrary assumptions are shown as to how quickly reaction rates could decrease per unit of residence time. Charts typical of FIG. 1A can be established experimentally for each specific algae species and specific photobioreactor configuration and operating environment. Actual reaction rates lines a, b, c depend on the probability that the respective reacting molecules will collide and on the fact that as products of reaction appear, in this case mainly oxygen and new algae, the probability of collisions among the reacting molecules decreases.

Once all of the needed reaction rate data are available, a further consideration for the design of the photobioreactor array is the minimization of the land area (footprint) that would be required by the photobioreactor arrays (102 in FIG. 1) to accommodate the captured carbon dioxide from /fossil energy production and from recycled carbon dioxide. The footprint would depend on the physical dimensions of the photobioreactor tube arrays and the number of modules required for given amounts of available by-product carbon dioxide production and recycled carbon dioxide.

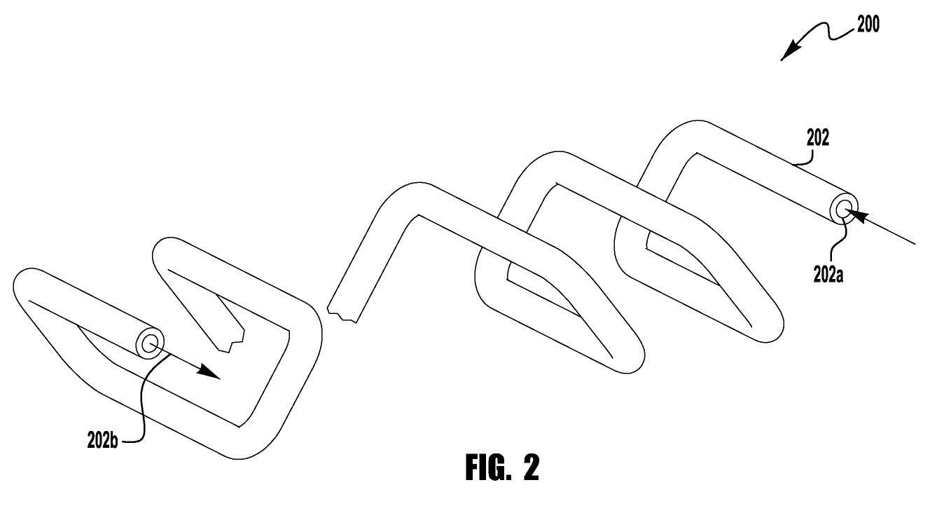

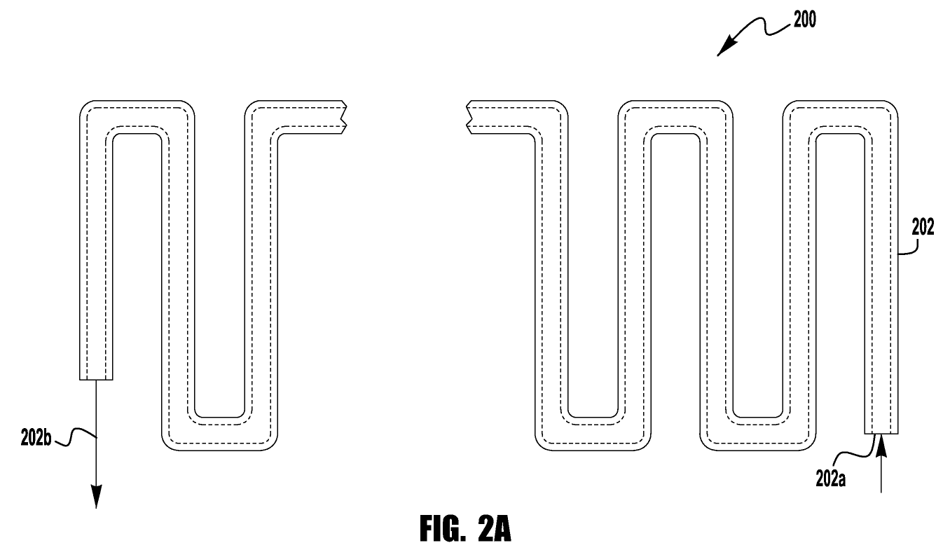

FIG. 2 shows an illustrative view of photobioreactor module configuration 200. The reactants within the enclosed environment of the photobioreactor module include recycled and by-product carbon dioxide from the power generating system, water, and algae, which circulate while being exposed to solar radiation within the photobioreactor. The reactants are charged at the entrance 202a of a tube 202 forming the photobioreactor module and the products leave from the departure exit 202b of tube 202. As noted above the total length of path through tube 202, its cross-sectional area, and the volumetric rate of flow of the reactants define the residence time and the extent to which the reactions complete. This invention embodies the prospect that additional rows of photobioreactor tubes can be included depending on the penetration of solar radiation (insolation) and use of supplemental artificial illumination.

FIG. 2A shows a top view of FIG. 2.

In an embodiment of the present invention, the artificial illumination can be provided by cultivating algae within appropriately-dimensioned photobioreactor arrays having internal artificial illumination.

A major consideration in determining the configuration of a photobioreactor module concerns the photons produced from the insolation, which represent the energy input required by the chemical reactions. i.e., by the photosynthesis that leads to the production of algae. The effective penetration of photons into the mass of the flowing reactants is limited, perhaps to as little as three centimeters. This means, especially if diameter of a photobioreactor tube exceeds three centimeters, that much of the flowing reactants do not react until mixing during the flow through the tube exposes them to photons. Reaction rates accordingly decrease. Nevertheless, the diameter of a photobioreactor tube should be much larger, especially for its structural strength.

Therefore, as shown in FIGS. 3A, 3B, and 3C (3A-3C), the photobioreactor tubes 200 in a module can be formed of a single length of tube 202 which repeatedly bends back on itself 180 degrees to form a continuous route to the extent required for the needed residence time, as shown, for example in FIG. 2A.

The tube 202 can be provided with a concentric inner tube 204 dimensioned to provide an annular area across which the reactants flow and into which insolation can penetrate from the outside and the photons provided by artificial illumination can penetrate from the center. Placing the inner tube 204 concentric with the outer tube 202 achieves a more uniform dissemination of photons across the flow path of the reactants.

Referring again to FIGS. 3A, 3B and 3C, three embodiments of a photobioreactor tube 202 are illustrated with different configurations of fluorescent tubing imbedded in the circular inner tube 204 disposed within and concentric with the outer photobioreactor tube 202. While fluorescent tubing is illustrated, it is within the terms of the disclosed embodiments to substitute other types of light sources such as for example “neon” tubing.

In the outer photobioreactor tubes 202 shown in FIGS. 3A, 3B, and 3C, the bundle of fluorescent or light emitting tubing 206, 208, 210, respectively, is imbedded in the inner tube 204 disposed concentrically within the photobioreactor outer tube 202. The difference in diameters D1 of outer tube 202 and D2 of the inner tube 204, which represents the annulus A1, is selected such the annulus A1 is about six centimeters in width, or larger if experimental findings justify, since irradiation occurs in both directions outward from the central tube 204 and inward from insolation through the outer tube 202.

The number of fluorescent tubes 212 can be symmetrically increased in increments of two up to the limit of availability of large diameter tubing for the photobioreactor module 200. As the number of fluorescent tubes increases so does the cross sectional area of the annulus A1, which increases the capacity for the photosynthesis reactions to accept increasing quantities of carbon dioxide, which then decreases the number of photobioreactor modules that would be needed to accommodate the quantity of captured carbon dioxide available.

The wavelengths of light represented by the photons should concentrate on the two ends of the visible spectrum. This distribution of wavelengths could be simulated for the fluorescent tubing by selection of the phosphors used and for “neon” type tubing by selection of the gases used. It appears that electrical input could be adjusted to vary the intensity of the radiation (number of photons per unit time) into the reactant mix of the selected algae species under conditions of essentially 100% carbon dioxide input and the presence of enzyme catalysts.

In a preferred embodiment, the addition of lighting in the photobioreactor tube 202 is illustrated with different configurations of fluorescent tubing to provide photon densities that can compensate for the variations in insolation during the day and for variations in insolation throughout the configuration of a photobioreactor module.

Depending on the penetration of insolation and artificial illumination, it should also be practical to consider two or more rows of photobioreactor tubing in the array beyond that as shown in FIG. 2. With the use of two or more rows of photobioreactor tubing, the land area (footprint) for a given system can be reduced significantly.

The light source for the emitting elements can be varied. As noted above, the two promising candidates are either fluorescent phosphors, such as is used in conventional fluorescent lighting or in the selection of gases in “neon” type tubing that concentrate on light emissions in the desired wavelengths.

Another embodiment, which also provides artificial illumination, is the use of light-emitting diodes (LEDs). FIG. 4 illustrates a structure for the incorporation of LEDs 204 that is analogous to the structures in FIG. 3 but differs in the nature of the illumination, their structural support, and their connections to the sources of electricity.

This embodiment includes an outer photobioreactor tube 202 with an inner concentric tube 204. LEDs can be disposed within the inner tube 204 as shown in FIG. 4. The LEDs can be an array of unit-sized LEDs 214 disposed around an electricity bus 216 and formed in a rectangular shape to fit the dimensions of the containing tube 204. With the LEDs 214 being in an hexagonal shape, as shown, the connecting wires 218 could be of the same physical shape or bent as necessary for all LED arrays.

In each of the structures shown in FIGS. 3A-3C and 4, a spacing of the outer tube is required to maintain the inner tubes 204 concentric with the outer tube 202 along its length. Further, perforated spacers (not shown) are needed to assure that the luminescent tubing support remains concentric along the length of the photobioreactor.

As in the discussion above, the dimensions of the annulus A1 can be determined for maximized cross-sectional area consistent with the ability of the insolation and artificial illumination to penetrate within the reactant mix.

The light-emitting chemicals in the diodes can be selected to result in radiation of a mix of frequencies at the ends of the visible spectrum, either as a single color for each diode in an alternating pattern, or as diodes that emit in a combination of the appropriate frequencies. Other structures are practical and this invention embodies variations that employ the same principles. For example, the supporting structure for the LEDs may be a hollow square of a non-conducting plastic material upon which the LED arrays are mounted and in which the electrical buses are internally incorporated. The individual arrays can then be electrically connected through the periodic spacers that maintain the concentricity. Four LED arrays can be mounted on the sides of the square and shaped to fit the circumference of the inner tube.

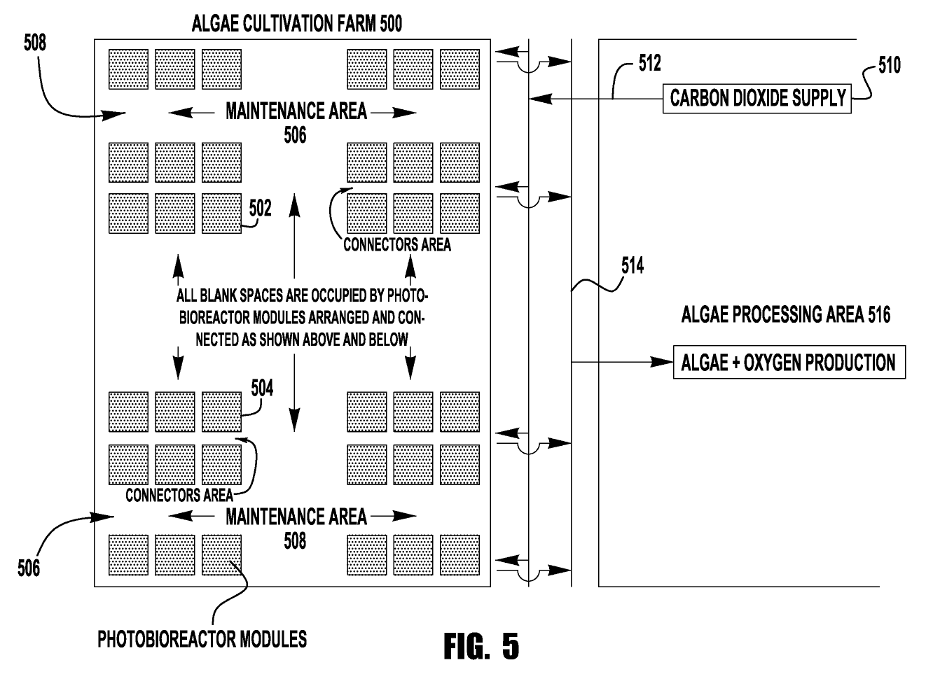

FIG. 5 illustrates how the algae cultivation farm 500 can be configured for the number of required photobioreactor modules 502 and 504 (compare photobioreactor module configuration 200, FIG. 2). The modules 502 are preferably interconnected and the modules 504 are preferably interconnected. The illustration of FIG. 5 illustrates maintenance areas 506 and 508, which provide space for the removal and reinstallation of the tubes of the photobioreactors as well as narrower spacing between rows of photobioreactor modules for the piping and electrical connections to the individual modules. The length of the arrays can be twenty four feet but this is only exemplary and any desired length can be mentioned for illustrative purposes. Carbon dioxide supply 510 enters the algae cultivation farm 500 from the fossil energy production installations (not seen) into line 512 and is distributed to the photobioreactor modules 502 and 504. The algae and oxygen production from the farm is collected from the rows of photobioreactors 514 and delivered to the algae processing area 516. The spaces between groups of photobioreactor modules are actually occupied by similar modules, but these are not shown for purposes of simplicity.

If desired, external artificial illumination can be provided by incorporating floodlighting over all, or any part of, the area occupied by the photobioreactor arrays (not shown). The wavelength distribution of the floodlight can be adjusted to focus on the red and blue ends of the visible spectrum.

### Fossil Energy Production Systems

FIG. 6 illustrates a view of the system 600 to continuously process carbon dioxide in an algae cultivation plant 14 for establishing synergism between fossil energy production and algae cultivation and processing. A coal gasification energy production system 602 is shown as a typical system with only components that are essential to the synergism. Coal gasification can also produce a range of alternative energy products other than electrical power as well as the by-product carbon dioxide. The coal gasification system 602 has oxygen, high pressure steam, coal, and algae oil introduced through lines 604, 606, 608, and 610, respectively. The oxygen, high pressure steam, coal and algae oil are directed through a line 612 into a coal gasifier 614. Oxygen from an air separation unit 616 is also directed into the coal gasifier 614 through a line 618, but, depending on the availability of oxygen from algae cultivation and processing system 14, this stream of oxygen 618 may either be unnecessary or decreased in quantity. Captured carbon dioxide is recovered within the system 602 and directed out of the system through a line 620 to line 151 of the Algae Cultivation and Processing system 14 as shown in FIG. 1. The by-product methane production 622 may be disposed, for example, either through marketing to a local natural gas pipeline installation or to blending with the fuel gas supply to the gas turbine in the coal gasification system when the product is electrical power.

In summary, the gasification plant 602 provides carbon dioxide for algae production. In return, it receives: (a) an oxygen stream; (b) a stream of algae oil to reduce the consumption of coal; and (c) a stream of methane to supplement and reduce the consumption of coal when the coal gasification system operates to produce electrical power.

FIG. 6 shows the linkages between an Algae Cultivation and Processing system 14 and a fossil energy gasification system 602 that embody the linkage principles of this invention.

A description of the linkages is the following: The carbon dioxide by-product, shown as produced in the lower Coal Gasification and Power Generation system 602 is produced at a pressure suited for conveyance to the Gas/Liquid Dispersion device 110 of the Algae Cultivation and Processing system 14. The carbon dioxide is produced continuously on a 24/7 basis except during periods of shutdown for maintenance. If a market exists for the by-product carbon dioxide, such as in enhanced oil recovery, not all of the carbon dioxide produced need be delivered to the Algae Cultivation and Processing system 14. For such a case, the CO₂ Compression Box (not shown) from coal gasification and power generation system 602 can be designed to provide the carbon dioxide for algae cultivation at the appropriate pressure.

The algae oil product shown as produced in the Algae Cultivation and Processing system 14 is delivered through line 106 to the Gasifier Section of the Coal Gasification Energy Production System 602. In a preferred embodiment, the algae oil will be used to slurry the pulverized coal feed, which slurry then will be fed to the coal gasifier 614 in appropriately designed burners together with the oxygen and steam mixture to the extent required to maintain the desired temperature in the gasifier. As such, the pulverized coal with algae oil not only replaces a conventional use of water-based pulverized coal slurry but also offers an increase in thermodynamic efficiency of the energy production cycle by eliminating the heat requirement for the evaporation of liquid water.

The oxygen product shown as produced in the Algae Cultivation and Processing system 14 is delivered to the Coal Gasifier 614 and can replace all, or in part, the oxygen requirement for the gasification process that is conventionally supplied by an air separation unit 616. Since the oxygen is produced in the photobioreactors at a low pressure, it can be compressed to the pressure required by the gasifier 614. Also, depending on the extent to which the photosynthesis reactions in the photobioreactors reach completion, the oxygen may have a content of carbon dioxide. This carbon dioxide content can react in the gasifier 614 with the carbon in the coal to produce carbon monoxide, which is desirable. As such, it decreases the amount of high-pressure steam required to maintain the temperature in the gasifier 614 at the desired level.

The incorporation of anaerobic digestion in the Algae Cultivation and Processing system 14 to process the oil-free algae for the production of methane (at location “A”) delivers all or part of the methane product to supplement the fuel to the gas turbine (not shown), if the system 602 is oriented to electrical power generation. This linkage has a beneficial effect in reducing the amount of excess air for combustion of high hydrogen-content gas turbine fuel in order to maintain inlet temperature to the first stage of the gas turbine expander section at below the maximum allowable. This linkage then serves to increase the thermal efficiency of power generation from the turbine The carbon dioxide jointly produced in anaerobic digestion is internally recirculated to the photobioreactors. Alternatively, the oil-free algae can be marketed as an animal feed supplement or mixed with the coal feed to the gasifier 614.

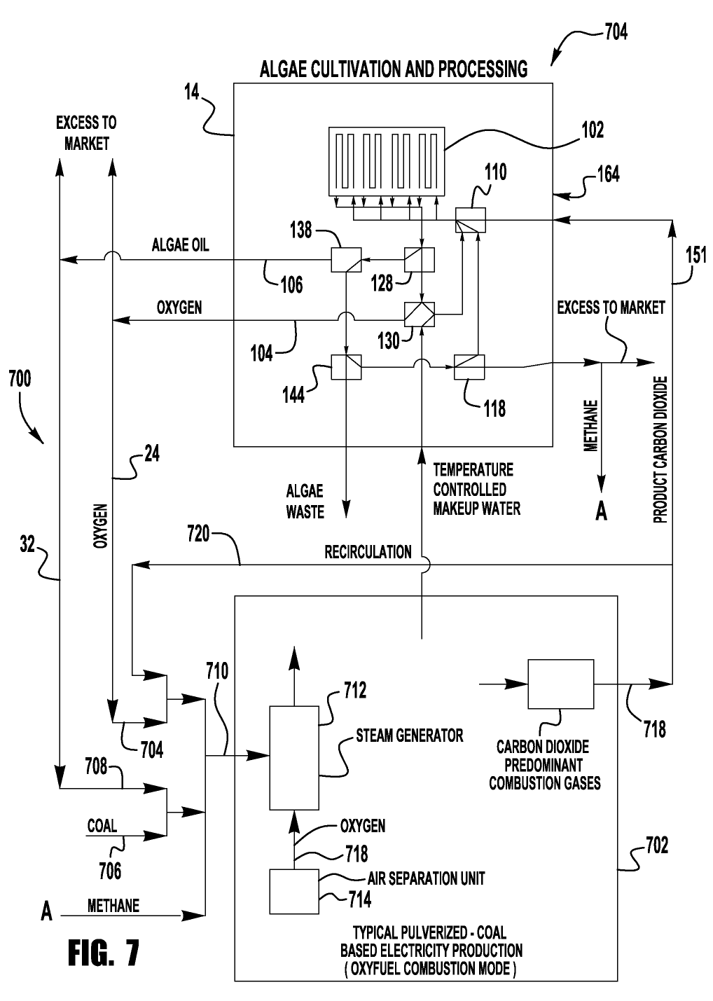

FIG. 7 illustrates a view of the system 700 for establishing synergism between an Oxyfuel combustion system 702 and an algae cultivation and processing system 14, as shown in FIG. 1. The relationship is the same as described above with regards to FIG. 6 in describing the relationship between the coal gasification energy production system 602 and the algae cultivation and processing system 14 except in FIG. 7 an Oxyfuel combustion system 702 is utilized instead of a coal gasification energy production system 602. Oxyfuel combustion system 702 is a simplified schematic illustration of how Oxyfuel combustion operates. In Oxyfuel combustion, the recirculation of the combustion gases results in a stream containing almost 100% carbon dioxide, which replaces the nitrogen in the combustion air normally used. The net input of carbon from the fuel is released as practically a pure carbon dioxide stream 151.

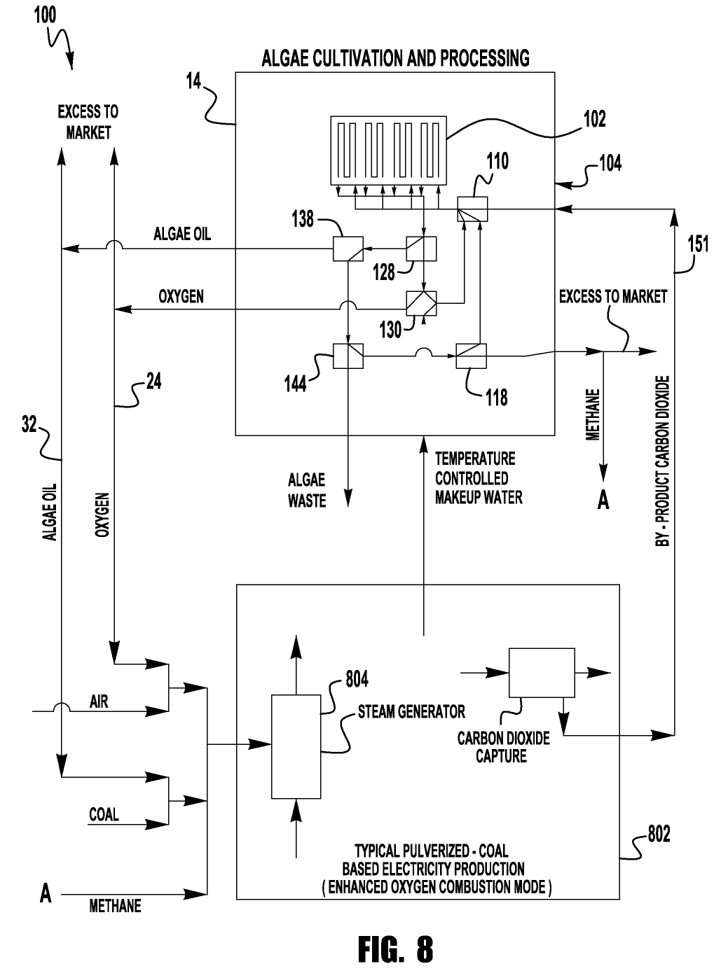

Normally for Oxyfuel combustion, the intention is that the volume percent of oxygen in admixture with the carbon dioxide will be the same as it is in air, about 21%. It is not necessary to limit the oxygen content to this level, but instead to increase it to the extent that the furnace in the steam generator can accommodate an increased temperature. This extent will be determined in part by the melting point of the ash in the coal fuel in furnace designs based on dry ash or higher temperatures in furnace designs based on the slagging of the ash by exceeding its melting point temperature. The net effect will be an increase in the thermal efficiency of the electrical power generating cycle (decrease in heat rate) for a given installation. This invention embodies this potential as enhanced-oxygen Oxyfuel combustion and complements the enhanced oxygen combustion mode described in connection with FIG. 8 below.

The Oxyfuel combustion system 702 has oxygen, coal and algae oil introduced through lines 704, 706, and 708, respectively. The oxygen, coal and algae oil are directed through a line 710 into a steam generator 712. Oxygen from an air separation unit 714 is also directed into the steam generator 712 through a line 716. Depending on the supply of oxygen 704, the need for the air separation unit 714 may either be reduced or eliminated. Captured carbon dioxide is recovered within the system 702 and directed out of the system through a line 718 to line 151 of the algae cultivation and processing system 14 as shown in FIG. 1. A portion of the carbon dioxide is diverted for recirculation through line 720 to substitute for the function of nitrogen in conventional combustion, which limits the temperatures in the steam generator furnace.

As previously discussed a major advantage of the algae cultivation and processing system 14 in combination with the Oxyfuel combustion system 702 will be achieved using the carbon dioxide generated by the system 702 of FIG. 7 continuously, i.e. twenty four hours seven days a week. In this case, artificial illumination can be introduced by the algae cultivation system 14, as described in FIGS. 3A, 3B, 3C, and 4, outside of daylight hours.

The preferred embodiments are based on recycling the algae oil produced in the algae cultivation and processing system 14 to the coal gasification energy production system 602 or to the Oxyfuel combustion power generating system 702, as shown in FIGS. 6 and 7 respectively, to replace some of the coal requirement and on recycling the by-product oxygen to replace a significant quantity of the oxygen requirement for coal gasification and for Oxyfuel combustion, which otherwise would have to be produced in an air separation plant. The function and structure of the preferred embodiments provide a synergistic relationship between a coal gasification energy production system 602 and alternatively an Oxyfuel coal combustion power generating system 702 and an algae cultivation and processing system 14 in which the fossil energy production system and the algae cultivation and processing system exchange carbon dioxide and oxygen as well as algae oil and algae biomass.

If the algae cultivation and processing system 14 includes anaerobic digestion of oil-free algae biomass, additional synergism is produced. Methane (essentially synthetic natural gas) is produced at “A” for recycling to replace some of the coal used in Oxyfuel combustion at “A”. The net effects in summary are then (a) a reduction of undesirable emissions of carbon dioxide to the atmosphere, (b) a reduction in, if not elimination of, the amount of oxygen required to be produced from air separation, (c) a reduction in coal consumption, and (d) an increase in the attractiveness of algae cultivation and processing because of the value of the oxygen, of methane, and of the algae oil it produces. Depending on the algae species and the conditions of operation in the photobioreactor 102, it is expected that the recoverable oil content in the algae could range from about 30% to 70% by weight.

FIG. 7 shows the linkages between an Algae Cultivation and Processing system 14 and a Oxyfuel combustion power generating system 702 that embody the linkage principles of this invention.

In Oxyfuel Combustion, the combustion gases are recirculated to replace the nitrogen in the combustion air at the burners. This recirculation linkage 720 is shown in FIG. 7. The oxygen itself is supplied from the algae cultivation and processing unit 704 and from an air separation unit 714 as the conditions may dictate. Once stabilization is reached, the composition of the recirculated gases is practically 100% carbon dioxide. Although this illustration is specific for the combustion of coal, the use of petroleum fuel oil or natural gas (if other than in the combine cycle mode) is equally applicable. A description of the linkages is the following:

(1) The excess carbon dioxide is delivered to the Algae Cultivation and Processing Box as shown in FIG. 7. If a market exists for part of this excess, such as in enhanced oil recovery, the marketable carbon dioxide would need to be compressed at a high pressure for delivery, since the carbon dioxide in Oxyfuel combustion is available only at slightly above atmospheric pressure. For use in the algae photobioreactors, the carbon dioxide would be compressed to the nominal pressure suited for control of its distribution to the individual photobioreactor modules.

(2) The algae oil product shown as produced in the Algae Cultivation and Processing system 14 is delivered to the steam generator 712 in the Oxyfuel and Power Generation system 702. This invention contemplates that the algae oil will be used to slurry the pulverized coal feed and that the slurry will be fed to the burners of the steam generator (not shown). The steam generator may be of conventional design or may be of circulating fluidized bed design.

(3) The oxygen product shown as produced in the Algae Cultivation and Processing system 14 is delivered to the Oxyfuel Combustion and Power Generation system 702 through line 704. It can replace all, or in part, the oxygen requirement for the gasification process that is conventionally supplied by the air separation unit 714. Also, depending on the extent to which the photosynthesis reactions in the photobioreactors reach completion, the oxygen may have a content of carbon dioxide. This carbon dioxide content can act to reduce the amount of carbon dioxide recirculation and essentially ultimately represent a recycle back to the Algae Cultivation and Processing system 14. In a preferable embodiment, the quantity of oxygen may increase beyond the 21% volume percent content in air, which can raise the temperature in the boiler furnace and likely decrease the heat rate of the power generation cycle. The limit can depend on not exceeding the ash fusion temperature in “dry-bottom” furnaces. Higher furnace temperatures can be feasible in “wet-bottom” furnaces in which ash-fusion temperatures are exceeded.

(4) The incorporation of anaerobic digestion in the Algae Cultivation and Processing system 14 to process the oil-free algae for the production of methane (at location “A”) delivers all or part of the methane product to supplement the fuel to the steam generator 712 in the Oxyfuel combustion system 702 at location “A”. The carbon dioxide jointly produced in anaerobic digestion is recirculated to the photobioreactors. Alternatively, the oil-free algae can be marketed as an animal feed supplement or mixed with the coal feed to the gasifier.

FIG. 8 illustrates the linkages between Algae Cultivation and Processing system 14 and a Conventional Pulverized-Coal Combustion and Power Generation system 802 that embody the linkage principles of this invention. Practically 100% content carbon dioxide is captured in this mode from combustion gases that can contain up to about 15% volume percent of carbon dioxide. The carbon dioxide is captured by introducing a processing unit in the combustion gases stream just before the chimney (not shown). The carbon dioxide is captured by the use of selected solvents operating in a regenerative mode for recovery and reuse of the solvents. The carbon dioxide capture process illustrated in FIG. 8 may be any of several that are commercially available. Although this illustration is specific for the use of coal, the use of petroleum fuel oil or natural gas (if other than in the combine cycle mode) is equally applicable. A description of the linkages is the following:

(1) The captured carbon dioxide is delivered to the Algae Cultivation and Processing system 14 as shown in FIG. 8 though line 151. If a market exists for the carbon dioxide, such as in enhanced oil recovery, part of the carbon dioxide can be compressed and diverted to this use. For use in the photobioreactors, the carbon dioxide is compressed to a pressure suited for control and distribution to the photobioreactor modules.

(2) The algae oil product shown as produced in the Algae Cultivation and Processing system 14 is delivered to the Steam Generator 804 in the Conventional Combustion Power Generation system 802. This invention intends that the algae oil can be used to slurry pulverized coal feed and that the slurry can be fed to the burners of the Steam Generator 804. The boiler may be of conventional design or may be of circulating fluidized bed design.

(3) The oxygen product shown as produced in the Algae Cultivation and Processing system 14 is delivered to the Conventional Combustion Power Generation system 802. The use of oxygen in this mode differs from the uses in the modes illustrated in FIGS. 6 and 7 above, since no air separation unit 616, 714 is employed for this mode. Instead, the linked oxygen is used to enhance the oxygen content of the combustion air to the extent that can be tolerated by the resulting furnace temperatures in dry-bottom or wet-bottom furnaces. The result is a reduction in the nitrogen content of the combustion gases and a reduction in the quantity of combustion gases. The inert gas load (nitrogen) that has to be accommodated by the carbon-capture unit is reduced with consequent reduction in capital cost of the unit and its consumption of electricity.

(4) The incorporation of anaerobic digestion in Algae Cultivation and Processing system 14 to process the oil-free algae for the production of methane (at location “A”) delivers all or part of the methane product to supplement the fuel to the steam generator 804 in the Conventional Pulverized Coal Combustion Power Generation system 802 at location “A”. The carbon dioxide jointly produced in anaerobic digestion is recirculated to the photobioreactors. Alternatively, the oil-free algae can be marketed as an animal feed supplement or mixed with the coal feed to the steam generator or gasifier.

### Other Embodiments

For the Gasification mode, the embodiments include the production of energy forms other than electricity from the synthesis gas that gasification produces. Accordingly, the linkages described for the gasification mode that produces electricity also apply to the production of alternative energy products such as those identified below. Chemical shifting, referred to below, involves the chemical reaction CO+H₂0→CO₂+H₂.

By chemically shifting the carbon monoxide in the synthesis gases completely to carbon dioxide, the result is the primary production of industrial hydrogen instead of electricity.

By chemically shifting the carbon monoxide in the synthesis gases to an appropriate ratio of carbon to hydrogen, the result is the production of petroleum-equivalent transportation fuels by a commercially-proven process called Fischer-Tropsch (F-T) synthesis.

By chemically shifting the carbon monoxide in the synthesis gases to an appropriate ratio of carbon to hydrogen, the result is the production of methane, which is the equivalent of natural gas of pipeline quality, through commercially-available catalytic processing.

By chemically shifting the carbon monoxide in the synthesis gases to an appropriate ratio of carbon to hydrogen, the result is the production of industrial methanol, a gasoline additive and an industrial raw material, through commercially-available catalytic processing.

If the configuration for a gasification system still allows for the use of an air separation unit, for the production of industrial hydrogen, for the availability of nitrogen from the air separation unit, and for the availability of by-product carbon dioxide, the production of urea fertilizer through commercially available processing.

For Algae Cultivation and Processing the embodiments described recognize that such cultivation could occur in varying climates especially in climates that represent wide variation between maximum and minimum ambient temperatures and that algae cultivation is best conducted within a narrow temperature range in the ambient region. Accordingly, this invention embodies facilities representing an additional linkage, wherein waste heat streams from fossil energy production can be linked to streams in the algaeculture system for temperature maintenance purposes and wherein electricity generated in the fossil energy system can be linked to refrigeration systems in the algaeculture and processing system for cooling streams for temperature maintenance purposes. For example, temperature-controlled makeup water 134 shown in FIG. 1 can be provided as a linkage between fossil energy production and algae cultivation and processing from the water demand in the fossil energy production system, either by heating from waste heat, or otherwise, streams within the system or by cooling from installed refrigeration systems within the system, as appropriate.

For all of the embodiments described herein variations in the distance between the algae cultivation and processing system and any of the three modes of fossil energy production systems can exist. Accordingly, the embodiments embody linkages over variable distances of separation such that interchanges continue as linkages as described in this invention through transportation in pipelines and/or railway or highway tank cars or vehicles.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

1. (canceled)

2. The system of claim 27 wherein:
the renewable energy production installation includes photobioreactor arrays for using the carbon dioxide from the fossil energy production system so as to produce algae from solar radiation and from complementary artificial illumination, when solar energy is unavailable or inadequate; and
the photobioreactor arrays forms an enclosed environment in which the carbon dioxide from the fossil energy production system, water, and algae circulates while being exposed to solar radiation within the photobioreactor arrays.

3. The system of claim 2 wherein the photobioreactor arrays have tubes complete with internal light emitting elements to create artificial illumination for continuous algae cultivation.

4. The system of claim 2 wherein the renewable energy production installation further includes:
an algae/liquid separation device to separate effluent from the photobioreactor arrays to algae biomass and recycled liquid;
an algae oil extractor to receive the algae biomass and recover the oil-content of the algae; and
a liquid recycle device to recover the oxygen from the recycled liquid produced in the algae/liquid separation device.

5. The system of claim 2 wherein the renewable energy production installation further includes piping for delivering the oil-content of the algae and the oxygen from the recycled liquid to the fossil energy production system and electrical connections to provide the electricity required for artificial illumination.

6. The system of claim 4 wherein the photobioreactor arrays have at least one tube provided with an inner tube to decrease the actual cross section through which the carbon dioxide, water, and algae circulate.

7. The system of claim 6 wherein light emitting elements within the photobioreactor arrays are disposed within the inner tube of at least one photobioreactor tube to provide enhanced dissemination of light photons throughout the cross section of the at least one tube.

8. The system of claim 7 wherein the inner tube is a concentrically placed circular tube in relation to the outer tube of the photobioreactor array.

9. The system of claim 7 wherein the light emitting elements are fluorescent or “neon” type lights.

10. The system of claim 7 wherein the light emitting elements are individual light emitting diodes (LEDs) arranged in supported arrays oriented to the circumference of the inner tube.

11. The system of claim 7 wherein the light emitting elements are selected so as to emphasize the emissions of light frequencies at both ends of the visible spectrum.

12. (canceled)

13. The system of claim 27 wherein the fossil energy production system is an Oxyfuel combustion system.

14. The system of claim 27 wherein the fossil energy production system is an oxygen enhanced combustion system.

15. The system of claim 27 in which the renewable energy production installation and the fossil energy production installation are physically separated such that interchanges between the installations continue as linkages through transportation in pipelines and/or railway or highway tank cars or vehicles.

16. The system of claim 27 in which the renewable energy production installation and the fossil energy production installation have linkages therebetween for cold climates such that temperature-controlled makeup water is supplied from the fossil energy system to the renewable energy production installation for providing a heat source in the photobioreactor arrays to maintain temperatures within the optimum range for algae cultivation.

17. The system of claim 27 in which the renewable energy production installation and the fossil energy production installation have linkages therebetween for warm climates such that temperature-controlled makeup water to the renewable energy production installation cools the reactants in the photobioreactor arrays in order to maintain temperatures within the optimum range for algae cultivation.

18-26. (canceled)

27. A system for establishing synergism between a renewable-energy production installation and a fossil-energy production installation, comprising:
the fossil-energy production system completely combusting a fossil fuel having a carbon content such that essentially the entire carbon content of the fossil fuel is converted to essentially pure carbon dioxide, and the mineral content of the fossil fuel is produced without any substantial melting or slagging;
means for capturing the essentially pure carbon dioxide from the fossil-energy production installation without its emission to the atmosphere and exporting the essentially pure carbon dioxide to the renewable-energy production installation;
the renewable-energy production installation having means for processing the essentially pure carbon dioxide from the fossil-energy production installation through the cultivation of algae by solar radiation (insolation) to algae biomass and by-product oxygen;
means within the renewable-energy production installation for processing the algae biomass to produce a combustible algae oil and a carbon-rich algae biomass residue;
means within the renewable-energy production installation for collecting the by-product oxygen and exporting at least a portion of it to the fossil-energy production installation;
means within the fossil-energy production installation to receive the exported by-product oxygen and to substitute at least a portion of atmospheric air for combusting the fossil fuel with the by-product oxygen; and
means within the renewable-energy production installation to export at least a part of the algae oil produced to the fossil-energy installation in order to replace an equivalent quantity of the fossil-fuel being combusted.

28. The system of claim 27 further including:
means in the fossil-energy installation known as Oxyfuel for capturing the carbon dioxide content of the combustion gases through recirculation of the combustion gases, for replacing nitrogen in the atmospheric air of the recirculated combustion gases, and replacing the oxygen in atmospheric air with the by-product oxygen from the renewable-energy installation to enable the complete combustion of the fossil fuel.

29. The system of claim 28 wherein the means in the fossil-energy installation further includes:
means for recirculating combustion gases produced from the complete combustion of the fossil fuel such that the composition of the recirculated combustion gases is essentially entirely carbon dioxide;
means for replacing the atmospheric air that enables the complete combustion of the fossil fuel by the by-product oxygen from the renewable-energy installation;
means for replacing the nitrogen in the atmospheric air by the recirculated combustion gases; and
means for processing the non-recirculated portion of the combustion gases produced by the complete combustion of the fossil fuel which otherwise exit to the atmosphere to provide the captured carbon dioxide.

30. The system of claim 27 further including:
means in the fossil-energy installation known as “enriched-air” combustion for replacing a portion of the nitrogen in the combustion air with the by-product oxygen produced in the renewable-energy installation;
means in the fossil-energy installation for capturing the carbon dioxide content of the combustion gases through absorption by a solvent or a chemical solution that can be regenerated by a change in the operating conditions of temperature and pressure;
means for capturing and exporting the captured carbon dioxide to the renewable-energy production installation in which the captured carbon dioxide is used for the cultivation of algae biomass and the production of by-product oxygen;
means for exporting at least a portion of the by-product oxygen of the renewable energy installation to the fossil-energy production installation to replace part of the atmospheric air employed for the complete combustion of fuel in the fossil-fuel production installation; and
means within the renewable-energy production installation for processing the algae biomass to extract its oil content and produce a carbon-rich algae biomass residue.

31. The system of claim 27 wherein the fossil fuel in the fossil-energy installation is a slurry of a solid fossil fuel and the algae oil exported from the renewable-energy installation.

32. The system of claim 27 wherein the complete combustion of the fossil fuel by the use of by-product oxygen imported from the renewable energy production installation includes:
means for increasing the proportion of oxygen in relation to either the recirculated combustion gases or the atmospheric air such that the mineral content of the fossil fuel is produced as a molten slag; and
means for removing the molten slag from the fossil-energy production installation.

33. The system of claim 27 further including:
means for providing temperature-controlled water through the incorporation of refrigeration technology within the renewable-energy production installation;
means for utilizing the temperature-controlled water for algae cultivation; and
means for utilizing the temperature-controlled water produced in the renewable-energy production installation in the fossil-energy production installation.

34. The system of claim 27 wherein:
the renewable-energy production installation includes means for digesting the carbon-rich algae-biomass residue and processing a resulting off gas of a mixture of carbon dioxide and methane to essentially pure methane; and
the fossil-energy production installation includes means for receiving this essentially pure methane and adding it to the fossil fuel.

35. The system of claim 27 wherein the renewable-energy production installation includes:
means for digesting algae-biomass residue, after extraction of the algae oil from the algae biomass, and processing a resulting off gas of a mixture of carbon dioxide and methane to essentially pure carbon dioxide; and
means for adding the essentially pure carbon dioxide for the cultivation of algae.

36. The system of claim 27 in which the exported bi-product oxygen mixed with either recirculated combustion gases or atmospheric air is used in excess of the stoichiometric requirement to avoid the presence of carbon monoxide in the combustion gases.
